(19) United States
(12) Patent Application Publication
(10) Pub. No.: US 2013/0261360 A1
(43) Pub. Date: Oct. 3, 2013

(54) METHODS AND APPARATUSES FOR PROCESSING RENEWABLE FEEDSTOCKS

(71) Applicants: Donald A. Eizenga, Elk Grove Village, IL (US); Daniel L. Ellig, Arlington Heights, IL (US)

(72) Inventors: Donald A. Eizenga, Elk Grove Village, IL (US); Daniel L. Ellig, Arlington Heights, IL (US)

(73) Assignee: UOP LLC, Des Plaines, IL (US)

(21) Appl. No.: 13/436,451

(22) Filed: Mar. 30, 2012

(51) Int. Cl. C07C 5/27; B01J 19/00; C07C 7/00

(52) U.S. Cl. 585/310; 422/187

(57) ABSTRACT

Methods and apparatuses for processing a renewable feedstock are provided herein. In an embodiment, a method for processing a renewable feedstock includes deoxygenating a stream of the renewable feedstock at a first pressure to form a stream of paraffins. The pressure of the stream of paraffins is reduced to a second pressure which is at least about 345 kPa less than the first pressure. Further, normal paraffins in the stream of paraffins are converted to form a stream of converted paraffins.

### TECHNICAL FIELD

The present invention generally relates to methods and apparatuses for processing renewable feedstocks, and more particularly relates to methods and apparatuses that deoxygenate renewable feedstocks at high pressure to form normal paraffins, and that isomerize or crack normal paraffins at low pressure to form fuel products.

### BACKGROUND

As the demand for diesel and jet boiling range fuel increases worldwide, there is increasing interest in feedstock sources other than petroleum crude oil. One such source is what has been termed “renewable” and “biological” feedstocks. These renewable biological feedstocks include, but are not limited to, plant oils such as corn, jatropha, camelina, rapeseed, canola, and soybean oil; algal oils; and animal fats such as tallow and fish oils. The common feature of these sources is that they are composed of glycerides and Free Fatty Acids (FFA). Both of these classes of compounds contain n-aliphatic carbon chains having from about 8 to about 24 carbon atoms. The aliphatic carbon chains in the glycerides or FFAs can be fully saturated or mono, di- or poly-unsaturated. The glycerides and FFAs in biological oils and fats can be converted into diesel or jet fuel using many different processes, such as hydro-deoxygenation and hydro-isomerization processes.

Fuel processed from renewable biological sources is desirable for a variety of reasons. Foremost, the use of renewable biological-sourced fuels reduces the demand for the extraction and use of fossil fuels. This is especially true for transportation fuels such as diesel and jet fuel. In addition to the ecological benefits of using biological-sourced fuel, there exists a market demand for such fuel. For fuel purchasers, the use of biological-sourced fuel can be promoted in public relations. Also, certain governmental policies may require or reward use of biological-sourced fuels.

However, renewable biological feedstocks present challenges in processing. For example, some renewable biological feedstocks are high in nitrogen. Elevated nitrogen levels in renewable biological feedstock streams render deoxygenation processing inefficient. Thus, there is a need for improved performance in processing renewable biological feedstocks for the production of fuels such as diesel and jet fuel.

Accordingly, it is desirable to provide methods and apparatuses for processing renewable biological feedstocks having elevated nitrogen levels under a first high pressure regime and a second low pressure regime. In addition, it is desirable to provide methods and apparatuses for deoxygenating renewable biological feedstocks at high pressure to form normal paraffins and for isomerizing the normal paraffins to isoparaffins at low pressure. Furthermore, other desirable features and characteristics will become apparent from the subsequent detailed description and the appended claims, taken in conjunction with the accompanying drawings and this background.

### BRIEF SUMMARY

Methods and apparatuses for processing a renewable feedstock are provided herein. In one exemplary embodiment, a method for processing a renewable feedstock includes deoxygenating a stream of the renewable feedstock at a first pressure to form a stream of paraffins. The pressure of the stream of paraffins is reduced to a second pressure which is at least about 345 kPa less than the first pressure. Further, normal paraffins in the stream of paraffins are converted to form a stream of converted paraffins.

In another exemplary embodiment, a method for processing a renewable feedstock includes deoxygenating a stream of the renewable feedstock at a first pressure of at least about 4140 kPa to form a stream of paraffins. In the method, the pressure of the stream of paraffins is reduced to a second pressure lower than the first pressure. The second pressure is no more than about 4820 kPa. Normal paraffins in the stream of paraffins are converted at the second pressure to form a stream of converted paraffins.

In a further exemplary embodiment, an apparatus for processing a renewable feedstock is provided. The apparatus includes a deoxygenation reactor configured to deoxygenate the renewable feedstock at a first pressure to form an effluent stream including paraffins. A separator is configured to remove a hydrocarbon fraction from the effluent stream comprising at least about 95 wt % paraffins. Further, the apparatus includes a means for reducing the pressure of the hydrocarbon fraction to a second pressure less than the first pressure. The apparatus is provided with a conversion reactor configured to convert paraffins in the hydrocarbon fraction at the second pressure to form a branched-paraffin-enriched stream. Also, the apparatus includes a product separator configured to remove a liquid hydrocarbon product from the branched-paraffin-enriched stream.

### BRIEF DESCRIPTION OF THE DRAWING

Exemplary embodiments will hereinafter be described in conjunction with the following drawing figures, wherein like numerals denote like elements, and wherein:

FIG. 1 is a schematic overview of an apparatus and method for processing renewable feedstocks in accordance with an exemplary embodiment;

FIG. 2 is a more detailed schematic view of the apparatus and method of FIG. 1 in accordance with an exemplary embodiment; and

FIG. 3 is a schematic view of an apparatus and method for processing renewable feedstocks in accordance with an exemplary embodiment.

### DETAILED DESCRIPTION

The following Detailed Description is merely exemplary in nature and is not intended to limit the apparatuses or methods for processing renewable feedstocks claimed herein. Furthermore, there is no intention to be bound by any theory presented in the preceding Background or the following Detailed Description.

The various exemplary embodiments contemplated herein are directed to methods and apparatuses for processing renewable feedstocks for the production of a hydrocarbon stream useful as diesel or jet boiling range fuel. The term renewable feedstock is meant to include feedstocks other than those obtained from petroleum crude oil. The renewable feedstocks that can be used in the methods and apparatuses contemplated herein include any of those which comprise glycerides, fatty acid alkyl esters (FAAE), and free fatty acids (FFA). Most of the glycerides will be triglycerides, but monoglycerides and diglycerides may be present and processed as well. Examples of these feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, mustard oil, cottonseed oil, jatropha oil, inedible tallow, yellow and brown greases, lard, train oil, fats in milk, fish oil, algal oil, sewage sludge, cuphea oil, camelina oil, curcas oil, babassu oil, palm kernel oil, crambe oil, fatty acid methyl esters, lard, and the like. Additional examples of renewable feedstocks include non-edible vegetable oils from the group comprising Jatropha curcas (Ratanjoy, Wild Castor, Jangli Erandi), Madhuca indica (Mohuwa), Pongamia pinnata (Karanji Honge), and Azadiracta indicia (Neem). The renewable feedstocks may include ratanjoy oil, wild castor oil, jangli oil erandi oil, mohuwa oil, karanji honge oil, neem oil, or any oil from a natural source or produced through microbial action. The glycerides, FAAEs and FFAs of the typical vegetable or animal fat contain aliphatic hydrocarbon chains in their structure which have about 8 to about 24 carbon atoms, with a majority of the fats and oils containing high concentrations of fatty acids with 16 and 18 carbon atoms.

Mixtures or co-feeds of renewable feedstocks and petroleum-derived hydrocarbons may also be used as the feedstock. Other feedstock components which may be used, especially as a co-feed component in combination with the above listed feedstocks, include spent motor oils and industrial lubricants; used paraffin waxes; liquids derived from the gasification of coal, biomass, or natural gas followed by a downstream liquefaction step such as Fischer-Tropsch technology; liquids derived from thermal or chemical depolymerization of waste plastics such as polypropylene, high density polyethylene, and low density polyethylene; and other synthetic oils generated as byproducts from petrochemical and chemical processes. Mixtures of the above feedstocks may also be used as co-feed components. In some applications, an advantage of using a co-feed component is the transformation of what may have been considered to be a waste product from a petroleum-based or other process into a valuable co-feed component to the current process.

Often, renewable feedstocks include elevated levels of nitrogen. It is believed that nitrogen is the most difficult heteroatom to hydrotreat due to catalyst surface interaction and/or steric hindrance. Because of the elevated nitrogen levels in renewable feedstock streams, typical deoxygenation processing is not efficient. However, it has been determined that complete or near complete deoxygenation processing of feedstocks having elevated nitrogen levels can be performed at higher pressures. The methods and apparatuses contemplated herein utilize an increased first stage pressure to perform sufficient deoxygenation on the renewable feedstock despite elevated levels of nitrogen.

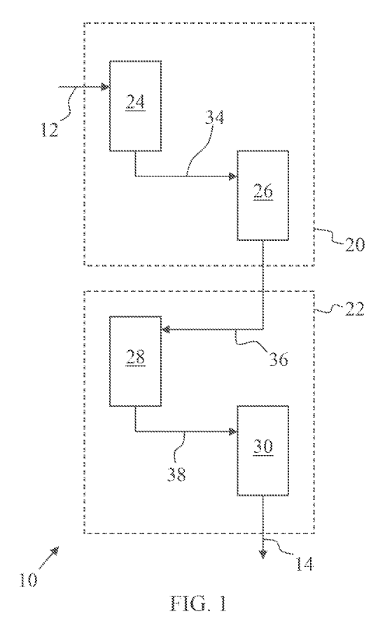

FIG. 1 generally illustrates an apparatus 10 for processing a renewable feedstock 12 to produce a hydrocarbon product stream 14 useful as a diesel or aviation fuel or blending component. As shown, the apparatus 10 includes an upstream first stage 20 that operates at a first pressure or in a first pressure range and a downstream second stage 22 that operates at a second pressure or in a second pressure range. The first stage 20 is provided with a deoxygenation zone 24 and a separator 26. The second stage 22 includes an isomerization and selective hydrocracking zone 28 and a product separator 30. In order to efficiently process the renewable feedstock 12 into a hydrocarbon product stream 14, the first stage 20 is operated at a higher pressure than the second stage 22.

In the apparatus 10, the first pressure in the first stage 20 is at least about 345 kilopascals (kPa) (about 50 psig) higher than the second pressure in the second stage 22. In an exemplary embodiment, the apparatus 10 is operated and controlled such that the first pressure is at least about 1380 kPa (about 200 psig) higher than the second pressure. In various exemplary embodiments, the first pressure is about 2070 kPa (about 300 psig), about 2760 kPa (about 400 psig), about 3450 kPa (about 500 psig), about 4140 kPa (about 600 psig), about 4820 kPa (about 700 psig), about 5520 kPa (about 800 psig), about 6890 kPa (about 1000 psig), about 8270 kPa (about 1200 psig), for example about 10340 kPa (about 1500 psig) higher than the second pressure.

In an exemplary embodiment, the first stage 20 is operated at a first pressure of at least about 3450 kPa to provide efficient deoxygenation; such as at least about 4140 kPa, at least about 4820 kPa, at least about 5520 kPa, at least about 6890 kPa, at least about 8270 kPa, at least about 10340 kPa, or at least about 13790 kPa. Further, the second stage 22 is operated at a second pressure to promote efficient isomerization and cracking. Typically, the second pressure is no more than about 6890 kPa. In exemplary embodiments the second pressure is no more than about 5520 kPa, no more than about 4820 kPa, no more than about 4140 kPa, no more than about 3450 kPa, no more than about 2760 kPa, for example no more than 2070 kPa.

As a result of processing by the apparatus 10, hydrocarbon products 14 may comprise diesel fuel products including hydrocarbons having boiling points in the diesel range. In certain embodiments, such diesel fuel products may be used directly as a fuel, may be blended with other components before being used as diesel fuel, or may receive additives before being used as a diesel fuel. Hydrocarbon products 14 comprising aviation fuel products include hydrocarbons having boiling points in the aviation range, which includes jet range, and may be used directly as aviation fuel or as a blending component to meet the specifications for a specific type of aviation fuel, or may receive additives before being used as an aviation fuel or blending component.

Depending upon the application, various additives may be combined with the aviation component or the diesel component generated in order to meet required specifications for different specific fuels. In particular, the aviation fuel composition generated herein complies with, is a blending component for, or may be combined with one or more additives to meet at least one of various national or international standards such as ASTM D7566 which provides specifications for Aviation Turbine Fuel containing Synthesized Hydrocarbons including up to 50 percent bioderived synthetic blending components—hydroprocessed esters and fatty acids (HEFA)—as additives to conventional jet fuel, ASTM D1655; DEF STAN 91-91; NATO codes F-35, F-34, and/or F-37; JP-8; JP-4; and JP-5, or the general grade requirements for Jet A, Jet A-1, Jet B, and TS-1 fuels as described in the IATA Guidance Material for Aviation Turbine Fuel Specifications. The aviation fuel is generally termed “jet fuel” herein and the term “jet fuel” is meant to encompass aviation fuel that meets the specifications above, and to encompass blending components of an aviation fuel meeting the specifications above. Additives may be added to the jet fuel in order to meet particular specifications. One fuel produced from glycerides or FFA as described herein is very similar to isoparaffinic kerosene or iPK, also known as a synthetic paraffinic kerosene (SPK) or synthetic jet fuel.

Renewable feedstocks 12 processed by the apparatus 10 may contain a variety of impurities. For example, tall oil contains esters and rosin acids in addition to FFAs. Rosin acids are cyclic carboxylic acids. The renewable feedstocks 12 may also contain contaminants such as alkali metals, e.g. sodium and potassium, phosphorous as well as solids, water and detergents. An optional first step, not shown in FIG. 1, is to remove as much of these contaminants as possible. One possible pretreatment step involves contacting the renewable feedstock 12 with an ion-exchange resin in a pretreatment zone at pretreatment conditions. The ion-exchange resin, such as an acidic ion exchange resin, can be used as a bed in a reactor through which the feedstock 12 is flowed through, either as upflow or downflow. Another technique includes contacting the renewable feedstock 12 with a bleaching earth, such as bentonite clay, in a pretreatment zone.

Another possible means for removing contaminants is a mild acid wash. This is carried out by contacting the renewable feedstock 12 with an acid such as sulfuric, nitric, phosphoric, or hydrochloric acid in a reactor. The acid and renewable feedstock 12 can be contacted either in a batch or continuous process. Contacting is done with a dilute acid solution usually at ambient temperature and atmospheric pressure. If the contacting is done in a continuous manner, it is usually done in a counter current manner. Yet another possible means of removing metal contaminants from the renewable feedstock 12 is through the use of guard beds which are well known in the art. These can include alumina guard beds either with or without demetallation catalysts such as nickel or cobalt. Filtration and solvent extraction techniques are other choices that may be employed.

As depicted in FIG. 1, the renewable feedstock 12 is passed to a deoxygenation zone 24 comprising one or more catalyst beds in one or more reactors. The term feedstock is meant to include feedstocks that have not been treated to remove contaminants as well as those feedstocks purified in a pretreatment zone or oil processing facility. In the exemplary deoxygenation zone 24, the feedstock 12 is contacted with a catalyst in the presence of hydrogen at hydrogenation conditions to hydrogenate the olefinic or unsaturated portions of the aliphatic hydrocarbon chains. The catalysts are any of those well known in the art, such as nickel or nickel/molybdenum dispersed on a high surface area support. Other possible catalysts include one or more noble metal catalytic elements dispersed on a high surface area support. Non-limiting examples of noble metals include Pt and/or Pd dispersed on gamma-aluminas Hydrogenation conditions typically include a temperature of about 200° C.. to about 450° C..

The catalysts enumerated above are also capable of catalyzing decarboxylation, decarbonylation, and/or hydrodeoxygenation of the feedstock 12 to remove oxygen. Decarboxylation, decarbonylation, and hydrodeoxygenation are herein collectively referred to as deoxygenation reactions. Deoxygenation conditions include a temperature of about 200° C.. to about 460° C.. with embodiments in the range of about 288° C.. to about 400° C.. Since hydrogenation is an exothermic reaction, as the feedstock flows through the catalyst bed the temperature increases and decarboxylation, decarbonylation, and hydrodeoxygenation will occur. Although the hydrogenation reaction is exothermic, some feedstocks may be highly saturated and not generate enough heat internally. Therefore, some embodiments may require external heat input. Thus, it is envisioned that all the reactions occur simultaneously in one reactor or in one bed, though typical operation will utilize multiple beds, and possibly multiple reactors. Alternatively, the conditions can be controlled such that hydrogenation primarily occurs in one bed and decarboxylation, decarbonylation, and/or hydrodeoxygenation occurs in a second or additional bed(s). If only one bed is used, it may be operated so that hydrogenation occurs primarily at the front of the bed, while decarboxylation, decarbonylation and hydrodeoxygenation occur mainly in the middle and back of the bed. Finally, desired hydrogenation can be carried out in one reactor, while decarboxylation, decarbonylation, and/or hydrodeoxygenation can be carried out in a separate reactor. However, the order of the reactions is not critical to the success of the process.

The reaction product 34 from the hydrogenation and deoxygenation reactions flows to and is separated by separator 26. The reaction product 34 will comprise both a liquid portion and a gaseous portion. The liquid portion comprises a hydrocarbon fraction comprising n-paraffins (normal, i.e., straight-chain, paraffins) and having a large concentration of paraffins in the 15 to 18 carbon number range, though different feedstocks will have different distributions of paraffins. Part of the liquid portion may be used as a hydrocarbon recycle to the deoxygenation zone 24. The remaining liquid hydrocarbon fraction 36 may be useful as a diesel fuel or blending component. For use as other fuels, such as aviation fuels or blending components which typically have a concentration of paraffins in the range of about 9 to about 15 carbon atoms, the hydrocarbon fraction 36 requires additional downstream processing. Additional downstream processing is generally preferred for the improvement of properties of the hydrocarbon fraction 36 even when used as diesel fuel or blending component.

The gaseous portion of the reaction product 34 from the deoxygenation zone 24 comprises hydrogen, carbon dioxide, carbon monoxide, water vapor, propane, nitrogen or nitrogen compounds, sulfur components such as hydrogen sulfide, and/or phosphorous components such as phosphine. While not expressly shown in FIG. 1, the reaction product 34 from the deoxygenation zone 24 may be conducted to a hot high pressure hydrogen stripper. One purpose of the hot high pressure hydrogen stripper is to selectively separate at least a portion of the gaseous portion of the effluent from the liquid portion of the effluent. As hydrogen is an expensive resource, the separated hydrogen can be recycled to the deoxygenation zone 24 to conserve costs. Also, failure to remove the water, carbon monoxide, and carbon dioxide from the hydrocarbon fraction 36 may result in poor catalyst performance in the second stage 22. Water, carbon monoxide, carbon dioxide, ammonia and/or hydrogen sulfide are selectively stripped in the hot high pressure hydrogen stripper using hydrogen. The hydrogen used for the stripping may be dry, and free of carbon oxides. The temperature may be controlled in a limited range to achieve the desired separation and the pressure may be maintained at approximately the same pressure as the deoxygenation zone 24 to minimize both investment and operating costs. The hot high pressure hydrogen stripper may be operated at conditions including a temperature of about 40° C.. to about 350° C.. or a temperature of about 50° C.. to about 350° C.

In such an embodiment, the reactor product 34 enters the hot high pressure stripper and at least a portion of the gaseous components is carried with the hydrogen stripping gas and separated into an overhead stream. The remainder of the deoxygenation zone effluent stream is removed as hot high pressure hydrogen stripper bottoms and contains the liquid hydrocarbon fraction having components such as normal hydrocarbons with from about 8 to about 24 carbon atoms. A portion of this liquid hydrocarbon fraction in hot high pressure hydrogen stripper bottoms can be used as a hydrocarbon recycle.

Generally, it is desirable to operate deoxygenation reaction zones at lower pressures because higher pressure operations are more costly to build and to operate as compared to their lower pressure counterparts. Nevertheless, the present methods and apparatuses provide for the high pressure regimes described above. It is noted that higher operating pressures may increase the prevalence of the deoxygenation reaction while reducing the prevalence of the decarboxylation reaction.

Because the hydrocarbon fraction 36 comprises essentially all normal paraffins, it will have poor cold flow properties. Many diesel and aviation fuels and blending components must have better cold flow properties and so the hydrocarbon fraction 36 is passed to the second stage 22 and further reacted in the isomerization and selective hydrocracking zone 28 under isomerization conditions to convert, i.e., isomerize and/or crack, at least a portion of the normal paraffins into converted paraffins, i.e., branched paraffins including isoparaffins. As discussed above, the second stage 22 is operated at a lower pressure than the first stage 20.

In the isomerization and selective hydrocracking zone 28, the hydrocarbon fraction 36 is contacted with an isomerization catalyst in the presence of hydrogen at isomerization conditions to isomerize the normal paraffins into branched paraffins. In some embodiments, only minimal branching is required, enough to overcome cold flow problems of the normal paraffins. In other embodiments, a greater amount of isomerization is desired. The predominant isomerization product is generally a mono-branched hydrocarbon. Along with the isomerization, some hydrocracking of the hydrocarbons will occur. The more severe the conditions of the isomerization zone 28, the greater the amount of hydrocracking of the hydrocarbons. The hydrocracking occurring in the isomerization zone 28 results in a wider distribution of hydrocarbons than resulted from the deoxygenation zone 24. Further, increased levels of hydrocracking produces higher yields of hydrocarbons in the aviation fuel boiling range.

The isomerization of the paraffinic hydrocarbons in the isomerization zone 28 can be accomplished in any manner known in the art or by using any suitable catalyst known in the art. Suitable catalysts comprise a metal of Group VIII (IUPAC 8-10) of the Periodic Table and a support material. Suitable Group VIII metals include platinum and palladium, each of which may be used alone or in combination. The support material may be amorphous or crystalline. Suitable support materials include aluminas, amorphous aluminas, amorphous silica-aluminas, ferrierite, laumontite, cancrinite, offretite, hydrogen form of stillbite, magnesium or calcium form of mordenite, and magnesium or calcium form of partheite, each of which may be used alone or in combination. Many natural zeolites, such as ferrierite, that have an initially reduced pore size can be converted to forms suitable for olefin skeletal isomerization by removing associated alkali metal or alkaline earth metal by ammonium ion exchange and calcination to produce the substantially hydrogen form. The isomerization catalyst may also comprise a modifier selected from the group consisting of lanthanum, cerium, praseodymium, neodymium, samarium, gadolinium, terbium, and mixtures thereof

The catalysts of the subject process can be formulated using industry standard techniques. It may be manufactured in the form of a cylindrical extrudate having a diameter of from about 0.8 mm to about 3.2 mm. The catalyst can be made in any other desired form such as a sphere or pellet. The extrudate may be in forms other than a cylinder such as the form of a well-known trilobe or other shape which has advantages in terms of reduced diffusional distance or pressure drop.

In general, isomerization conditions include a temperature of about 150° C.. to about 450° C.., such as above about 300° C., and below about 400° C. or below about 360° C. Other operating conditions for the isomerization zone are well known in the art, and the specific operating conditions used are predetermined and are dependant upon the desired product specifications and relative yields of the products.

The catalysts suitable for the isomerization of the paraffinic hydrocarbons and the conditions of the isomerization zone also operate to cause some hydrocracking of the hydrocarbons. Therefore, in addition to paraffinic hydrocarbon fractions suitable for use as diesel fuel or blending component, paraffinic hydrocarbons suitable for use as an aviation fuel or blending component may be additionally or alternatively generated. As illustrative of this concept, a concentration of paraffins formed from renewable feedstocks typically has about 15 to 18 carbon atoms, but additional paraffins may be formed to provide a range of from about 8 to about 24 carbon atoms. While a portion of the normal paraffins are isomerized to branched paraffins, the carbon number range of paraffins will not change with isomerization alone. However, some hydrocracking will occur concurrently with the isomerization, generating paraffins having boiling points from about 150° C. to about 300° C., which is lower than that of the majority of C15 to C18 paraffins produced in the deoxygenation reaction zone 24. The fraction having a boiling point range of about 150° C. to about 300° C. meets many aviation fuel specifications and can therefore be separated from the other boiling point ranges after the isomerization zone 28 in order to produce an aviation fuel. This will lower the overall yield of diesel fuel but allows the production of two fuel products: a diesel fuel and an aviation fuel.

The process severity in the isomerization zone 28 controls the potential yield of product for aviation fuel, the amount of light products that are not useful for diesel fuel or aviation fuel, and the isomerized/normal ratio of both aviation and diesel range fuel. Hydrocracking is controlled through catalyst choice and reaction conditions in an attempt to restrict the degree of hydrocracking. Ideally, each paraffin molecule would experience only a single hydrocracking event and ideally that single hydrocracking event would result in at least one paraffin in the C9 to C15 carbon number range. Careful choice of catalyst and control of the process conditions in the isomerization zone 28 both maximizes paraffin products in the aviation fuel range while minimizing the production of the light paraffins, i.e., paraffins with carbon chains of 3 or fewer, that are not useful for either diesel fuel or aviation fuel applications.

It is noted that fuel specifications are typically not based upon carbon number ranges. Instead, the specifications for different types of fuels are often expressed through acceptable ranges of chemical and physical requirements of the fuel. Often a distillation range from 10 percent recovered to a final boiling point is used as a key parameter defining different types of fuels. The distillations ranges are typically measured by ASTM Test Method D86 or D2887. Therefore, blending of different components in order to meet the specification is quite common While the aviation fuel product of the present process may meet aviation fuel specifications, it is expected that some blending of the product with other blending components may be required to meet the desired set of fuel specifications. The desired aviation fuel product is a highly paraffinic distillate fuel component having a paraffin content of at least 75% by volume.

As shown in FIG. 1, an isomerization effluent stream 38 obtained after all reactions have been carried out is passed to the product separator 30 and processed through one or more separation steps to obtain at least one purified hydrocarbon product stream 14, such as one useful as a diesel fuel or blending component or as an aviation fuel or blending component. A lighter stream of components not useful as diesel or aviation fuel, such as hydrocarbons with carbon chains of 3 or fewer carbons, may also separated.

The effluent stream 38 of the isomerization and selective hydrocracking zone 28 comprises both a liquid component and a gaseous component, various portions of which may be recycled, multiple separation steps may be employed. For example, hydrogen may be first separated in an isomerization effluent separator with the separated hydrogen being removed in an overhead stream. Suitable operating conditions of the isomerization effluent separator include, for example, a temperature of about 60° C. to about 100° C. If there is a low concentration of carbon oxides, or if the carbon oxides are removed, the hydrogen may be recycled back to the hot high pressure hydrogen stripper for use both as a rectification gas and to combine with the remainder as a bottoms stream. The remainder may be passed to the isomerization reaction zone 28 and thus the hydrogen may become a component of the isomerization reaction zone feed stream in order to provide the necessary hydrogen partial pressures for the reactor. Hydrogen is also a reactant in the deoxygenation zone 24, and different feedstocks 12 will consume different amounts of hydrogen. Furthermore, at least a portion of the remainder or bottoms stream of the isomerization effluent separator may be recycled to the isomerization reaction zone 28 to increase the degree of isomerization.

The remainder of the isomerization effluent after the removal of hydrogen still has liquid and gaseous components and may be cooled, by techniques such as air cooling or water cooling and passed to a cold separator where the liquid component may be separated from the gaseous component. Suitable operating conditions of the cold separator may include, for example, a temperature of about 20° C. to about 60° C. A water byproduct stream may also be separated. At least a portion of the liquid component, after cooling and separating from the gaseous component, may be recycled back to the isomerization zone 28 to increase the degree of isomerization. Prior to entering the cold separator, the remainder of the isomerization and selective hydrocracking zone effluent 38 may be combined with the hot high pressure hydrogen stripper overhead stream, and the resulting combined stream may be introduced into the cold separator.

The liquid component of the effluent stream 38 contains the hydrocarbons useful as diesel fuel and aviation fuel, termed diesel fuel range hydrocarbons and aviation fuel range hydrocarbons, respectively, as well as smaller amounts of naphtha and liquefied petroleum gas (LPG). The liquid component of the effluent stream 38 is purified in the product separator 30, such as a fractionation zone which separates lower boiling components and dissolved gases into an LPG and naphtha stream; an aviation range product; and a diesel range product. Suitable operating conditions of the product fractionation zone include a temperature of from about 20° C. to about 300° C. at the overhead. The conditions of the distillation zone may be adjusted to control the relative amounts of hydrocarbon contained in the aviation range product stream and the diesel range product stream.

The LPG and naphtha stream may be further separated in a debutanizer or depropanizer in order to separate the LPG into an overhead stream, leaving the naphtha in a bottoms stream. Suitable operating conditions of this unit would include a temperature of from about 20° C. to about 200° C. at the overhead. The LPG may be sold as valuable product or may be used in other processes such as a feed to a hydrogen production facility. Similarly, the naphtha may be used in other processes, such as the feed to a hydrogen production facility.

In another embodiment, the product separator 30 may comprise a single fraction column that operates to provide four streams, with the hydrocarbons suitable for use in a diesel fuel removed from the bottom of the column, hydrocarbons suitable for use in an aviation fuel removed from a first side-cut, hydrocarbons in the naphtha range being removed in a second site-cut and the propane and light ends, such as hydrocarbons having carbon chains or 3 or fewer carbons, being removed in an overhead from the column. In yet another embodiment, the product separator 30 may include multiple fractionation columns, with a first fractionation column separating the hydrocarbons useful in diesel and aviation fuels into a bottoms stream, and propane, light ends, and naphtha into an overhead stream. A second fractionation column may be used to separate the hydrocarbons suitable for use in a diesel fuel into a bottoms stream of the column and hydrocarbons suitable for use in an aviation fuel into an overhead stream of the column, while a third fractionation column may be employed to separate the naphtha range hydrocarbons from the propane and light ends. Also, dividing wall columns may be employed. The operating conditions of the one or more fractionation columns may be used to control the amount of the hydrocarbons that are withdrawn in each of the streams as well as the composition of the hydrocarbon mixture withdrawn in each stream. Typical operating variables well known in the distillation art include column temperature, column pressure, reflux ratio, and the like. The result of changing column variables, however, is only to adjust the vapor temperature at the top of the distillation column. Therefore the distillation variables are adjusted with respect to a particular feedstock in order to achieve a temperature cut point to give a product that meets desired properties.

Optionally, a portion of diesel-range hydrocarbons may be separated and recycled to the deoxygenation reaction zone 24. The hydrocarbon recycle stream may be taken from the reaction product 34 after the deoxygenation zone 24 and recycled back to the deoxygenation zone 24. Or the hydrocarbon recycle stream may be taken from the effluent of the separation unit 26, such as a hot high pressure separator. A portion of a hydrocarbon stream taken from, for example, a hot high pressure separator or a cold high pressure separator, may also be cooled down if necessary and used as cool quench liquid between the beds of the deoxygenation zone 24 to further control the heat of reaction and provide quench liquid for emergencies. The hydrocarbon recycle stream may be introduced to the inlet of the deoxygenation reaction zone 24 and/or to any subsequent beds or reactors. One benefit of the hydrocarbon recycle is to control the temperature rise across the individual beds. Operating with high hydrocarbon recycle and maintaining high levels of hydrogen in the liquid phase helps dissipate hot spots at the catalyst surface in the deoxygenation zone 24 and reduces coking and catalyst deactivation.

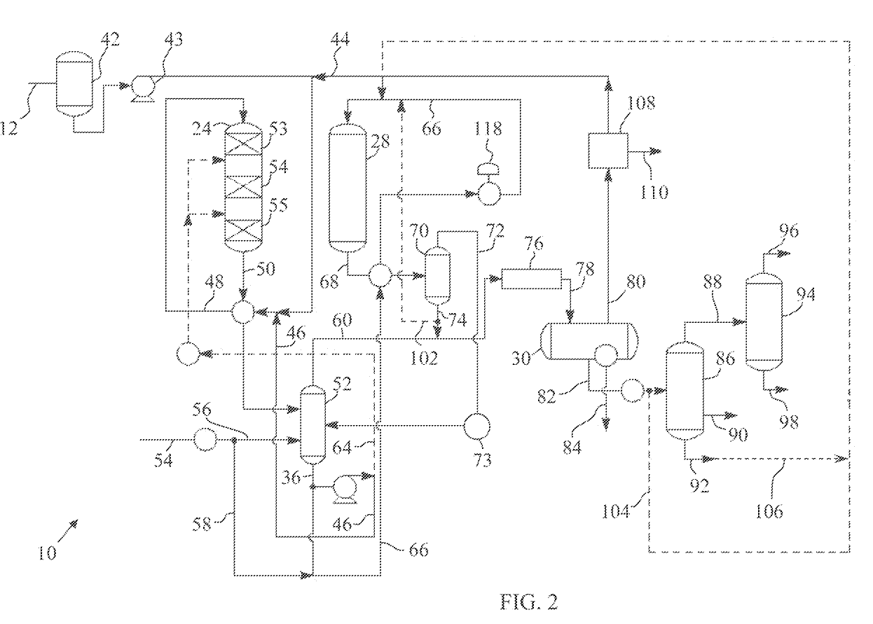

Turning to FIG. 2, a more comprehensive schematic of the apparatus 10 is provided. As shown, the renewable feedstock stream 12, which may pass through an optional feed surge drum 42 and pump 43, is combined with recycle gas stream 44 and recycle stream 46 (both discussed in more detail below) to form combined feed stream 48. The combined feed stream 48 is heat exchanged with reactor effluent 50 and is then introduced into deoxygenation reactor 24. The heat exchange may occur before or after the recycle 46 is combined with the feed 12. The deoxygenation reactor 24 may contain multiple beds shown as 53, 54, 55 and contains at least one catalyst capable of catalyzing decarboxylation and/or hydrodeoxygenation of the feedstock 12 to remove oxygen. Deoxygenation effluent stream 50, containing the products of the decarboxylation and/or hydrodeoxygenation reactions, is removed from deoxygenation reactor 24 and heat exchanged with combined feed stream 48 containing feed to the deoxygenation reactor 24. Deoxygenation effluent stream 50 comprises a liquid component containing largely normal paraffin hydrocarbons in the diesel boiling range and a gaseous component containing largely hydrogen, vaporous water, carbon monoxide, carbon dioxide and propane.

Deoxygenation effluent stream 50 is directed to hot high pressure hydrogen stripper 52. Make-up hydrogen 54 is divided into two portions, stream 56 and stream 58. Make-up hydrogen 56 is introduced to hot high pressure hydrogen stripper 52. In hot high pressure hydrogen stripper 52, the gaseous component of deoxygenation reactor effluent 50 is selectively stripped from the liquid component of deoxygenation reactor effluent 50 using make-up hydrogen 56. The dissolved gaseous component comprising hydrogen, vaporous water, carbon monoxide, carbon dioxide and at least a portion of the propane, is selectively separated into hot high pressure hydrogen stripper overhead stream 60. The remaining liquid component of deoxygenation reactor effluent 50 comprising primarily normal paraffins having a carbon number from about 8 to about 24 with a cetane number of about 60 to about 100 is removed as hot high pressure hydrogen stripper bottom or hydrocarbon fraction 36.

A portion of hydrocarbon fraction 36 forms recycle stream 46 and is combined with renewable feedstock stream 12 to create combined feed 48. Another portion of hydrocarbon fraction 36, optional stream 64, may be routed directly to deoxygenation reactor 24 and introduced at interstage locations such as between beds 53 and 54 and or between beds 54 and 55 in order, for example, to aid in temperature control. The remainder of hydrocarbon fraction 36 is combined with hydrogen stream 58 to form combined stream 66 which is routed to isomerization and selective hydrocracking reactor 28. Stream 66 may be heat exchanged with isomerization reactor effluent 68.

The product of the isomerization and selective hydrocracking reactor 28 containing a gaseous portion of hydrogen and propane and a branched-paraffin-enriched liquid portion is removed from reactor 28 as isomerization effluent 68. After an optional heat exchange with combined stream 66, the isomerization effluent 68 is introduced into hydrogen separator 70. Hydrogen separator 70 forms an overhead stream 72 containing primarily hydrogen, which may be recycled back to hot high pressure hydrogen stripper 52. As shown, stream 72 is compressed by compressor 73 to raise its pressure from the second stage pressure to the first stage pressure. The hydrogen separator 70 also forms bottom stream 74, which is air cooled using air cooler 76 and is introduced into the product separator 30 as cooled stream 78. In the product separator 30, the gaseous portion of the cooled stream 78, comprising hydrogen, carbon monoxide, hydrogen sulfide, carbon dioxide and propane, is removed in stream 80. The liquid hydrocarbon portion of the cooled stream 78 is removed in stream 82. A water byproduct stream 84 may also be removed from product separator 30.

In FIG. 2, the liquid hydrocarbon stream 82 is introduced to product stripper 86 where components having higher relative volatilities are separated into stream 88, components within the boiling range of aviation fuel are removed in stream 90, and the remaining diesel range components are withdrawn from product stripper 86 in stream 92. Stream 88 is introduced into fractionation unit 94 which operates to separate LPG into overhead 96 leaving a naphtha bottoms 98. Any of optional lines 102 (from the bottom stream 74 of hydrogen separator 70), 104 (from the liquid hydrocarbon stream 82), or 106 (from the diesel stream 92) may be used to recycle at least a portion of the isomerization zone effluent back to the isomerization reactor 28 to increase the amount of n-paraffins that are isomerized to branched paraffins.

The vapor stream 80 from product separator 30 contains the gaseous portion of the isomerization effluent comprising at least hydrogen, carbon monoxide, hydrogen sulfide, carbon dioxide and propane. As shown in FIG. 2, the vapor stream 80 is directed to an amine absorber so that carbon dioxide may be separated from the vapor stream 80. Because of the cost of hydrogen, it is desirable to recycle the hydrogen to deoxygenation reactor 24, but it is not desirable to circulate the carbon dioxide. In order to separate carbon dioxide from the hydrogen, the vapor stream 80 is passed through an amine absorber, also called a scrubber in zone 108. The amine chosen to be employed in amine absorber zone 108 is capable of selectively removing carbon dioxide. Exemplary suitable amines include a promoted or activated methyldiethanolamine (MDEA). The carbon dioxide is absorbed by the amine while the hydrogen passes through amine absorber zone 108 and into recycle gas stream 44 to be recycled to the deoxygenation zone 24. The amine is regenerated and the carbon dioxide is released and removed in line 110. Within the amine absorber zone 108, regenerated amine may be recycled for use again. Conditions for the absorber zone 108 include a temperature in the range of 30° C. to 60° C. The absorber zone 108 is operated at a temperature that is at least 1° C. higher than that of the separator 30. Keeping the absorber zone 108 warmer than the separator 30 operates to maintain any light hydrocarbons, such as those having carbon chains of 3 or more carbons, in the vapor phase and prevents the light hydrocarbons from condensing into the absorber solvent.

As noted in relation to FIG. 1, the first stage 20, including the deoxygenation zone 24 and separator 26, is operated at the first pressure while the second stage 22, including the isomerization zone 28 and separator 30, is operated at the second pressure lower than the first pressure. While various process schemes, flow paths and restrictions may be utilized to provide the desired pressure schemes, in an exemplary embodiment a control valve controls the flow of combined stream 66, which includes hydrocarbon fraction 36. Specifically, the control valve 118 is utilized to reduce the pressure of the combined feed 66 as it flows from the first stage 20 to the second stage 22 (shown in FIG. 1).

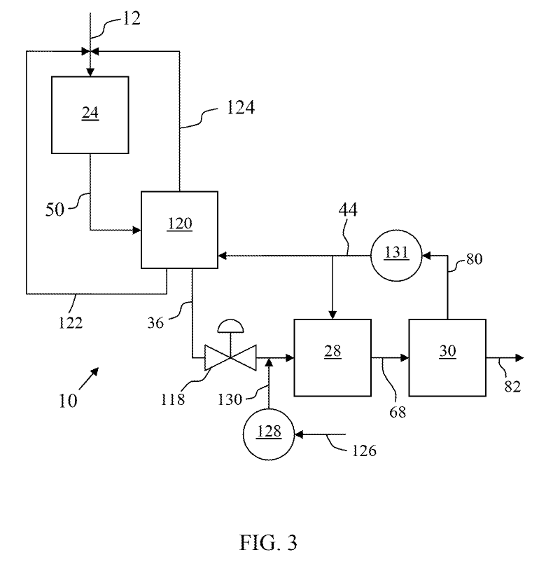

FIG. 3 is a simplified schematic of an alternative apparatus 10 with control valve 118. In FIG. 3, the feed 12 flows to the deoxygenation zone 24. The deoxygenation effluent 50 containing normal paraffins, water, carbon dioxide and propane exits the deoxygenation zone 24 and is fed to a separator/stripper zone 120. The separator/stripper zone 120 may include a hot separator with an enhanced hot stripper, a multi-stage fractionation unit, a distillation system, or similar known apparatus. In any event, the separator/stripper zone 120 removes the water, carbon dioxide, and propane from the deoxygenated effluent 50 in the form of a recycle liquid 122 and a recycle gas 124. In an exemplary embodiment, the recycle liquid 122 includes more than about 98 weight percent (wt %) paraffinic hydrocarbons and less than about 2 wt % hydrogen, water and light hydrocarbons, i.e., hydrocarbons having carbon chains of three or fewer carbons. In an exemplary embodiment, the recycle gas 124 comprises more than about 80 mole percent (mol %) hydrogen and less than about 20 mol % carbon oxides and light hydrocarbons. As shown, the recycle liquid 122 and recycle gas 124 are recycled and mixed with the feedstock 12 upstream of the deoxygenation zone 24 to improve process efficiency in the deoxygenation zone 24.

The hydrocarbon fraction 36 is formed by the removal of the recycle liquid 122 and recycle gas 124 from the deoxygenated effluent 50. In an exemplary embodiment, the hydrocarbon fraction 36 is formed of more than about 95 wt % paraffinic hydrocarbon, such as about 99.9 wt % paraffinic hydrocarbon, and less than about 0.2 wt % hydrogen, light hydrocarbon, and trace contaminants. As shown, the hydrocarbon fraction 36 flows through the control valve 118 which permits the upstream apparatuses including the deoxygenation zone 24 to operate at high pressure conditions while apparatuses downstream of the control valve 118 may operate at lower pressures. Specifically, the control valve 118 is configured to reduce the pressure of the hydrocarbon fraction 36 by at least about 345 kPa, and, in certain embodiments, may reduce the pressure of the hydrocarbon fraction by at least about 1380 kPa, about 2070 kPa, about 2760 kPa, about 3450 kPa, about 4140 kPa, about 4820 kPa, about 5520 kPa, about 6890 kPa, about 8270 kPa, or about 10340 kPa.

As stated above, fuel properties, such as cold flow properties, of a product liquid processed in apparatus 10 may be improved by converting normal paraffins into branched or isoparaffins in a desired range. Two of the main processes used to perform this conversion are cracking and isomerization. In cracking, high molecular weight fractions and catalysts are heated to the point where the carbon-carbon bonds break. Products of the reaction include paraffins of lower molecular weight than were present in the original fraction. In the isomerization process, normal paraffins, i.e., straight chain paraffins, are converted into branched chain isomers, which have improved cold flow properties. Typically, some isomerization occurs during the cracking process, which further improves cold flow properties of the fuel, including cloud point, cold filter plugging point, and pour point. These cold flow properties typically measure a fuel's ability to flow at colder temperatures.

In FIG. 3, the control valve 118 controls the flow of the hydrocarbon fraction 36 to the isomerization zone 28. As shown, make-up/recycle gas 126 (which may comprise hydrogen stream 58 of FIG. 2) is added to hydrocarbon fraction 36. The make-up/recycle gas 126 may be compressed to a desired pressure by a compressor 128 to form a compressed make-up gas stream 130 that is mixed with the hydrocarbon fraction 36 before being fed to the isomerization zone 28. The isomerization zone 28 isomerizes or cracks the normal paraffins to form the isomerization effluent 68 containing isoparaffins.

The isomerization effluent 68 is fed to the separator 30 which separates a vapor stream 80 from a liquid hydrocarbon stream 82. The vapor stream 80 may be compressed by a compressor 131 and fed as a recycle gas stream 44 to the isomerization zone 28 and/or the separator/stripper zone 120 as desired. Liquid hydrocarbon stream 82 may be utilized as a product liquid or processed further as indicated in FIG. 2.

Regardless of the exact design and structure of the separator/stripper zone 120 and control valve 118, the apparatus 10 is provided with the ability to operate upstream deoxygenation processing at the desired first pressure while operating the downstream paraffin conversion processing at the desired second pressure. Various valves and compressors are provided and arranged to enable the optimized flow and recycling of streams. As a result, renewable feedstocks can be processed into product liquids such as diesel or jet fuel.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the claimed apparatuses and methods for processing renewable feedstocks in any way. Rather, the foregoing detailed description will provide those skilled in the art with a convenient road map for implementing an exemplary embodiment. It being understood that various changes may be made in the function and arrangement of elements described in an exemplary embodiment without departing from the scope of the methods and apparatuses as set forth in the appended claims.

1. A method for processing a renewable feedstock comprising the steps of:
deoxygenating a stream of the renewable feedstock at a first pressure to form a stream of paraffins;
reducing the pressure of the stream of paraffins to a second pressure, wherein the second pressure is at least about 1380 kPa less than the first pressure; and
converting normal paraffins in the stream of paraffins to form a stream of converted paraffins.

2. The method of claim 1 further comprising:
decarboxylating the stream of the renewable feedstock concurrently with deoxygenating the stream of the renewable feedstock, wherein decarboxylating forms carbon dioxide and deoxygenating forms water; and
removing the carbon dioxide and water from the stream of paraffins.

3. The method of claim 1 further comprising separating a recycle gas from the stream of paraffins and recycling the recycle gas to the stream of the renewable feedstock.

4. The method of claim 1 further comprising separating a recycle gas from the stream of paraffins and recycling the recycle gas to the stream of the renewable feedstock, wherein the recycle gas comprises more than about 80 mol % hydrogen and less than about 20 mol % carbon oxides and light hydrocarbon.

5. The method of claim 1 further comprising separating a recycle liquid from the stream of paraffins and recycling the recycle liquid to the stream of the renewable feedstock.

6. The method of claim 1 further comprising separating a recycle liquid from the stream of paraffins and recycling the recycle liquid to the stream of the renewable feedstock, wherein the recycle liquid comprises more than about 98 wt % paraffinic hydrocarbon and less than about 2 wt % water, hydrogen, and light hydrocarbon.

7. The method of claim 1 wherein converting normal paraffins creates a gas stream, and further comprising separating the gas stream from the stream of converted paraffins and forming a product liquid from the stream of converted paraffins.

8. The method of claim 7 further comprising:
pressurizing the gas stream and feeding the pressurized gas stream to an enhanced hot separator;
delivering the stream of paraffins to the enhanced hot separator; and
using the pressurized gas stream to separate a recycle gas and a recycle liquid from the normal paraffins in the stream of paraffins.

9. The method of claim 1 wherein deoxygenating comprises deoxygenating the stream of the renewable feedstock at a pressure from about 4140 kPa to about 10340 kPa.

10. The method of claim 1 wherein reducing comprises reducing the pressure of the stream of paraffins to a pressure of from about 1380 kPa to about 4820 kPa less than the first pressure.

11. (canceled)

12. The method of claim 1 wherein reducing comprises reducing the pressure of the stream of paraffins to the second pressure, wherein the second pressure is at least about 2760 kPa less than the first pressure.

13. A method for processing a renewable feedstock comprising the steps of:
deoxygenating a stream of the renewable feedstock at a first pressure of at least about 4140 kPa to form a stream of paraffins;
reducing the pressure of the stream of paraffins to a second pressure lower than the first pressure, wherein the second pressure is no more than about 4820 kPa and wherein the second pressure is at least about 1380 kPa less than the first pressure; and
converting normal paraffins in the stream of paraffins at the second pressure to form a stream of converted paraffins.

14. The method of claim 13 further comprising:
decarboxylating the stream of the renewable feedstock concurrently with deoxygenating the stream of the renewable feedstock, wherein decarboxylating forms carbon dioxide and deoxygenating forms water; and
removing the carbon dioxide and water from the stream of paraffins.

15. The method of claim 13 further comprising:
separating a recycle gas from the stream of paraffins and recycling the recycle gas to the stream of the renewable feedstock; and
separating a recycle liquid from the stream of paraffins and recycling the recycle liquid to the stream of the renewable feedstock.

16. The method of claim 13 wherein deoxygenating comprises deoxygenating the stream of the renewable feedstock at the first pressure to form the stream of paraffins, wherein the first pressure is at least about 5520 kPa.

17. The method of claim 13 wherein deoxygenating comprises deoxygenating the stream of the renewable feedstock at the first pressure to form the stream of paraffins, wherein the first pressure is at least about 6890 kPa.

18. The method of claim 13 wherein deoxygenating comprises deoxygenating the stream of the renewable feedstock at the first pressure to form the stream of paraffins, wherein the first pressure is at least about 8270 kPa.

19. The method of claim 13 wherein reducing comprises reducing the pressure of the stream of paraffins to the second pressure, wherein the second pressure is at least 2070 kPa less than the first pressure.

20. (canceled)
